(19) United States
(12) Patent Application Publication
(10) Pub. No.: US 2016/0331922 A1
(43) Pub. Date: Nov. 17, 2016

(54) DISPOSABLE CONTAINER FOR AIR HYDRATION

(71) Applicant: John W. HOLADAY, Bethesda, MD (US)

(72) Inventor: John W. HOLADAY, Bethesda, MD (US)

(21) Appl. No.: 15/107,825

(22) PCT Filed: Dec. 22, 2014
(86) PCT No.: PCT/US14/71941

§ 371 Date: Jun. 23, 2016

Related U.S. Application Data
(60) Provisional application No. 61/920,379, filed on Dec. 23, 2013.
(60) Provisional application No. 62/018,100, filed on Jun. 27, 2014.

(51) Int. Cl. A61M 16/16; A61L 2/10; A61K 31/445; B65D 71/00; A61M 11/00

(52) U.S. Cl. CPC A61M 16/16; B65D 71/00; A61M 11/005; A61K 31/445; A61L 2/10; A61M 2205/123

(57) ABSTRACT

The present technology relates to a device and method to provide distilled water in pre-filled, disposable containers for use with humidifiers in assisted breathing units, such as continuous positive airway pressure (CPAP) devices. The technology also relates to methods for delivery of medications and inhalational and/or aromatic therapies through heated hydration chambers in CPAP devices and/or through nebulizers to be used to hydrate air in conjunction with CPAP devices. The present technology further relates to convenient multi-packs of pre-filled, disposable water containers to be provided in conjunction with CPAP devices.

### TECHNICAL FIELD

The present technology relates to a device and method to provide distilled water in pre-filled, disposable containers for use with humidifiers in assisted breathing units, such as continuous positive airway pressure (CPAP) devices. The technology also relates to methods for delivery of medications and inhalational and/or aromatic therapies through heated hydration chambers in CPAP devices and/or through nebulizers to be used to hydrate air in conjunction with CPAP devices. The present technology further relates to convenient multi-packs of pre-filled, disposable water containers to be provided in conjunction with CPAP devices.

### BACKGROUND OF TECHNOLOGY

Obstructive sleep apnea (OSA) is characterized by recurrent upper airway obstruction resulting in breathing cessation (apnea) or reduction of airflow during sleep (hypopnea). The periodic cessation of breathing during sleep is caused by tracheal muscle relaxation, which results in the narrowing and closing of the patient's airway. This narrowing of the trachea causes the person to repeatedly awaken during sleep, leading to chronic sleep deprivation. Chronic snoring is an indicator of OSA. Apneusis (cessation of breathing for various intervals) caused by OSA is diagnosed by monitoring patient's breathing patterns during their sleep. As many as 25% of people suffer from OSA. Failure to treat OSA is associated with significant cardiovascular, metabolic and central nervous system disorders, and these debilities of OSA are particularly exacerbated with aging. Present standards of care for OSA involve oral devices to bring the lower jaw forward, thus opening the airway, or the use of continuous positive airway pressure (CPAP) machines where positive airway pressure is applied to overcome airway obstructions and prevent the associated apnea and snoring.

Respiratory treatment apparatuses involves the delivery of a pressurized breathable gas, such as air, oxygen enriched air, or oxygen, to a patient's airways using a conduit and patient interface device. Gas pressures employed typically range from 4 cm H₂O to 30 cm H₂O, at flow rates of up to 180 L/min (measured at the mask), depending on patient requirements. For CPAP the pressurized gas acts as a pneumatic splint for the patient's airway in a CPAP device, preventing airway collapse, especially during the inspiratory phase of respiration.

Assisted breathing units, such as CPAP devices, provide therapy to a sleep apnea patient by delivering positive pressure to the airway, keeping it open when the tracheal muscles relax. Most CPAP devices include a pressure source, electronic circuitry to control the pressure source, and tubing connected to a mask worn by the patient. The CPAP pressure source, connective tubing and mask create a closed circuit for airflow between the patient's airways and the CPAP device. During treatment, a supply of pressurized gas is typically supplied to the patient through a patient interface, such as a nasal, oral or combination nasal/oral mask. The continuous flow of air from such CPAP devices can be irritating to the tissues of the nose, mouth and/or throat of the patient. This irritation may result in nosebleeds, increased mucous, congestion, and coughing or sneezing.

To help alleviate the drying effect of the pressurized air, manufacturers of CPAP machines have engineered hydrating attachments that volatilize water by heating, and the air is delivered over the vapors to become hydrated, and then to the patient through a flexible tube. These hydrating chambers typically involve permanent or disposable plastic containers for holding the water, with an aluminum base that sits on a heating element. The plastic containers have pathways that divert the air from the CPAP machines over the heated water to the tube that carries the hydrated air to the patient.

Several hundred milliliters of water is poured into the hydrating chamber before bedtime use of the CPAP machine. Distilled water is normally recommended to be used in CPAP devices since tap water and some bottled waters contain minerals and other components that dry out in the hydrating machine. These contaminants bond together and stick on the surfaces of the humidifier water tanks, providing a substrate for the growth of bacteria and molds. Continued contamination of the system will cause irreparable damage requiring the CPAP equipment to be replaced. To prevent contamination and to keep the CPAP devices in good working order, distilled water should be used nightly in the CPAP devices.

The advantages of incorporating humidification of the air supply to a patient are known, and respiratory apparatuses are known which incorporate humidifying devices. Such respiratory apparatuses commonly have the ability to alter the humidity of the breathable air or oxygen to reduce drying of the patient's airway and consequent patient discomfort and associated complications. The use of a humidifier unit placed between the flow generator and the patient mask produces humidified gas that minimizes drying of the nasal mucosa and increases patient airway comfort. In addition in cooler climates, warm air applied generally to the face area in and about the mask is more comfortable than cold air.

Many humidifier types are available, including humidifiers that are either integrated with or configured to be coupled to the relevant respiratory apparatus. If integrated within the relevant respiratory apparatus the humidifier is generally formed in a separate portion of the apparatus to the blower to prevent water from entering the blower. While passive humidifiers can provide some relief, generally a heated humidifier is required to provide sufficient humidity and temperature to the air so that the patient will be comfortable. Humidifiers typically comprise a water container having a capacity of several hundred milliliters, a heating element for heating the water in the container, a control to enable the level of humidification to be varied, a gas inlet to receive gas from the flow generator, and a gas outlet adapted to be connected to a patient conduit that delivers the humidified gas to the patient's mask.

Typically, the heating element is incorporated in a heater plate, which sits under and is, in thermal contact with the water container.

CPAP devices have been known to cause several issues for users. Some of these issues include allergies, infections, and inadequately filtered particulates from the delivered air. Also, a CPAP device with a humidifier unit requires frequent water replacement and cleaning. In some cases, daily water replacement is recommended for the CPAP device with a humidifier. Such a rigorous schedule of cleaning and water replacement can be tedious for most users. However, when the rigorous schedule is not maintained, the water in the CPAP machine may stagnate and allow for bacteria growth. The CPAP device may create an environment for bacterial growth that subjects the user to sinus infections, upper airway infections, conjunctivitis, and ear infections. The CPAP device also usually requires frequent filter changes that can be tedious and costly for a user. A lack of frequent filter changes, and a lack of bacterial/viral filtration, however, can lead to serious health implications since the CPAP device supplies air directly to the patient. It is vitally important that air is properly filtered and disinfected so that allergens, viruses, and bacteria are not introduced to the patient by the CPAP device.

Patients using heated humidifiers without regard to good hygienic practices in maintaining their humidifiers by thorough cleaning and replacing their breathing tubes showed a dramatic increase in upper airway infections, compared with those who cared for their equipment regularly.

Patients with OSA being treated with CPAP devices fitted with humidifiers may be aerosolizing bacteria, putting them at risk for developing respiratory infections. In a study reported in the Journal of Clinical Sleep Medicine, Vol. 3, No. 7 (2007), bacteria was recovered in breathing tubes of CPAP devices fitted with heated humidifiers with water contaminated with Brevundimonas diminuta or Serratia marcescens.

Nebulizers are commonly used for delivery of inhaled medications since they transform a liquid medication into a mist that can be comfortably and easily inhaled by a patient. The mist consists of a suspension of many miniscule liquid droplets in air and is created by the nebulizer rapidly, forcibly, and repeatedly disrupting the surface tension of the water and throwing droplets from the bulk liquid into the air. There are two types of nebulizers commonly used for inhalation therapy: the jet nebulizer and the ultrasonic nebulizer.

Jet nebulizers use a narrow stream of pressurized air to disrupt the surface tension of the bulk liquid in order to aerosolize the liquid medication. The average droplet size formed by jet nebulizers is between 5 and 600 μm, depending on the nozzle used (Hickey, A. J. 1996, Inhalation Aerosols: Physical and Biological Basis for Therapy. New York, N.Y.: Marcel Dekker). A jet nebulizer creates a thick mist allowing the flow of the air to carry the aerosolized medication to the patient. According to Gessler et al. 2001, Ultrasonic Versus Jet Nebulization of Iloprost in Severe Pulmonary Hypertension. Euro Resp., 17(1): 14-9, the efficiency of jet nebulizers in delivering aerosolized medication to a patient's lungs is approximately 36% to 42%. There are other disadvantages of using the jet nebulizer, however, the jet nebulizer is an inexpensive alternative.

Ultrasonic nebulizers achieve a higher efficiency of drug delivery to the patient than that achieved by jet nebulizers. Ultrasonic nebulizers include a piezoelectric crystal component that oscillates when an electric current is applied. The oscillations generate ultrasonic waves that move through the bulk liquid before disrupting the surface tension of the liquid, and cause the liquid medication to aerosolize into droplets. The oscillation of the piezoelectric component occurs at a frequency within the range of 1.0 to 4.0 MHz. Droplet size formed from ultrasonic nebulizers is between 3 and 6 μm. This size range for droplets is more clinically effective than the larger droplets generated by jet nebulizers. (Hickey, 1996). The efficiency of ultrasonic nebulizers at delivering aerosolized medication to the patient's lungs has been clinically shown to be in the range of 81% to 91%. (Gessler et al., 2001). Ultrasonic nebulizers are more expensive than jet nebulizers. Overall, ultrasonic nebulizers are more efficient at delivering medication and generate smaller droplets of aerosolized medication.

Non-invasive ventilation (NIV) is currently a form of standard care for patients suffering from respiratory insufficiency, sleep apnea, chronic obstructive pulmonary disease (COPD) and more severe acute and chronic respiratory failure. A common form of NIV is non-invasive positive pressure ventilation (NPPV) in which a mask or other interface supplies positive pressure flow to the nose and mouth. For less severe respiratory insufficiency and support, low-flow therapy (LFT) through a nasal cannula is common practice. In addition, high-flow therapy (HFT) has recently been introduced in which air or blended oxygen is preconditioned with heat and water vapor (humidity) to allow continuous delivery through a nasal cannula up to flow rates of 40 L/min. This approach is currently being applied to treat conditions such as pulmonary edema, COPD, bronchiectasis, and acute respiratory distress syndrome (post-intubation).

Patients receiving NIV typically have underlying respiratory and systemic conditions that can be effectively treated with a range of drugs administered non-invasively as pharmaceutical aerosols. However, both in vivo and in vitro studies have illustrated that high drug aerosol deposition losses occur in NIV tubing and delivery systems, resulting in very low delivery efficiencies on the order of <1-7% in both adults and children. Aerosol drug delivery to the lungs via NIV also employs conventional drug delivery devices (e.g. nebulizers and metered dose inhalers) that generate aerosols with relatively large particle sizes (3-5 μm). This large aerosol particle size results in high delivery system and nasal losses during NIV and may result in high variability in the amount of drug aerosol reaching the lungs. This is especially problematic for therapeutic substances with narrow therapeutic indices, and in fact, NIV may unfortunately not be appropriate for many next-generation medications, some of which have relatively narrow therapeutic windows. Moreover, high variability in delivery rates impacts the assessment of clinical trial results since the actual dose reaching a patient cannot be consistently established. However, despite low efficiency and associated problems, this current standard of care is often preferable to the alternative of temporarily halting NIV therapy for 10-30 minutes up to 2-8 times per day for administration of essential nebulized medications.

There is a need for the convenience of pre-filled, disposable water containers for use in CPAP devices. There is also a need for delivery of medications and inhalational and/or aromatic therapies through heated hydration chambers in CPAP devices and/or through nebulizers to be used in conjunction with CPAP devices.

### SUMMARY OF THE TECHNOLOGY

The present invention comprises a disposable container pre-filled with preferably distilled water for placement within the hydrating chamber of a continuous positive airway pressure (CPAP) device. The disposable containers can be packaged in a convenient storage pack wherein a disposable container can be removed from the storage pack as needed. The disposable container can also be in the form of bottled disposable water in convenient volumes and packages. The disposable containers and/or bottled water can optionally contain aromatic substances and therapeutic agents. The use of the disposable container thereby avoids the problem of infection from using non-disposable containers that are improperly cleaned between uses. The present invention also comprises the use of a nebulizer to hydrate CPAP air in lieu of a heated hydration chamber, and the use of the nebulized air delivery system to provide aromatic substances and/or therapeutic agents.

Thus, it is an object of the present invention to provide a pre-filled, disposable distilled water container designed to fit in the hydrating chamber of an assisted breathing apparatus.

It is another object of the present invention to provide a pre-filled, disposable distilled water container fabricated from a heat-resistant material.

It is yet another object of the present invention to provide a convenient supply of individual pre-filled, disposable distilled water containers and/or bottles of approximately 200-400 ml each.

Another aspect of the invention relates to pre-filled, disposable distilled water containers configured and arranged to deliver water vapor to one or more gas flow paths defined by the housing of the humidifier unit of CPAP devices.

In another aspect of the present invention inhalation therapies are added to the pre-filled, disposable distilled water containers prior to placement in CPAP devices.

In yet another aspect of the present invention aromatherapies are added to the pre-filled, disposable distilled water containers prior to placement in CPAP devices.

Another aspect of the present invention is to provide a convenient supply, such as a six-pack or other multi-unit packs, of pre-filled, disposable distilled water containers and/or bottles in conjunction with the CPAP device.

Further, the present invention includes the addition of non-volatile antiseptic to prevent the build up of bacteria or fungi in the hydrating chamber of a CPAP device.

It is also envisioned in the present invention to provide pre-filled disposable water containers for use in conjunction with a nebulizer.

Another aspect of the present invention is the use of a nebulizer to hydrate CPAP air in lieu of a heated hydration chamber, and the use of the nebulized air delivery system to provide aromatic substances and/or therapeutic agents.

Other aspects, features, and advantages of this technology will become apparent from the following detailed description when taken in conjunction with the accompanying drawings, which are a part of this disclosure and which illustrate, by way of example, principles of this technology.

### BRIEF DESCRIPTION OF THE DRAWINGS

The accompanying drawings facilitate an understanding of the various examples of this technology. In such drawings:

FIG. 1 depicts the hydrating chamber of a ResMed CPAP device in which the present invention in its various embodiments may be implemented.

FIG. 2 depicts the hydrating chamber of a Respironics CPAP device in which the present invention in its various embodiments may be implemented.

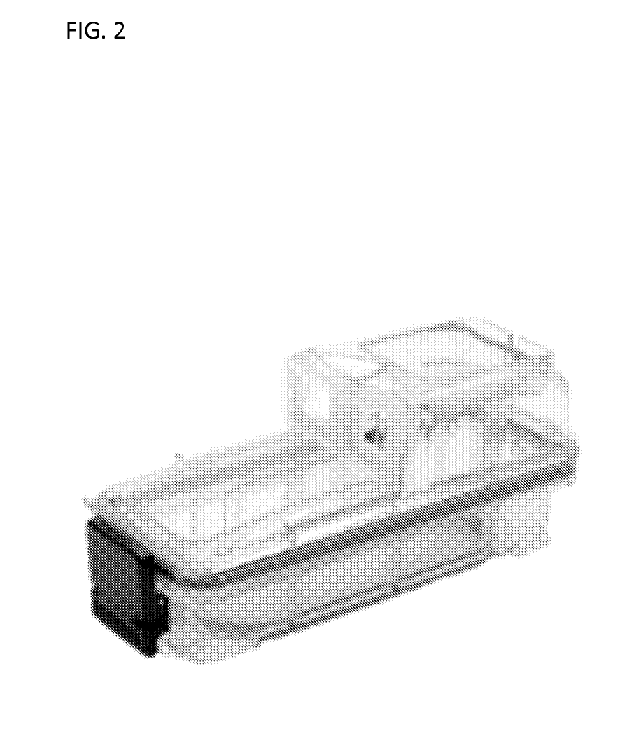

FIG. 3 depicts an ultrasonic nebulizer in which the present invention in its various embodiments may be implemented.

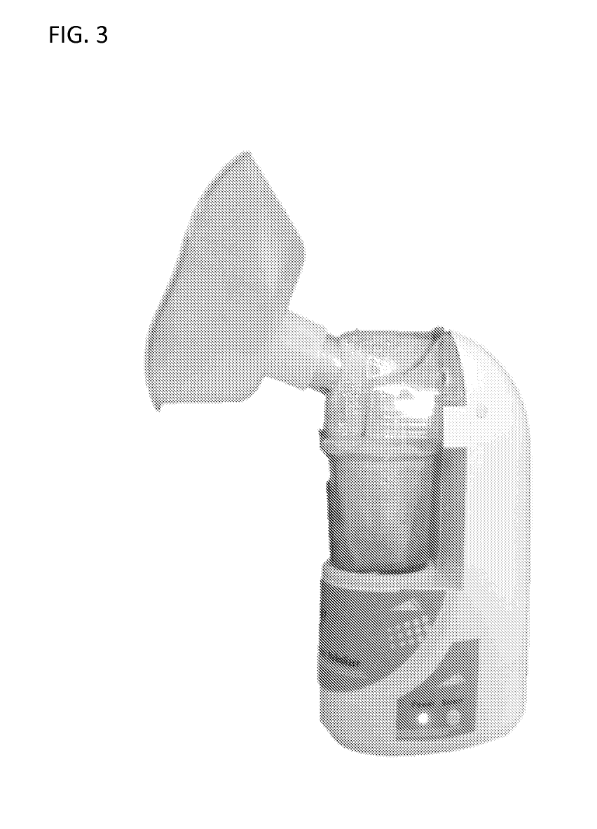

### DETAILED DESCRIPTION OF ILLUSTRATED EXAMPLES

The functionality of the various example embodiments will be explained in more detail in the following description, read in conjunction with the figures illustrating the example embodiments. Turning now to the drawings, example embodiments are described in detail.

As used herein, the singular form of “a”, “an”, and “the ” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The term “air” will be taken to include breathable gases, for example air with supplemental oxygen.

The subject headings used in the detailed description are included only for the ease of reference of the reader and should not be used to limit the subject matter found throughout the disclosure or the claims. The subject headings should not be used in construing the scope of the claims or the claim limitations.

The present invention addresses shortcomings of the prior art by providing pre-filled, disposable distilled water containers for use in a humidification system for a CPAP device.

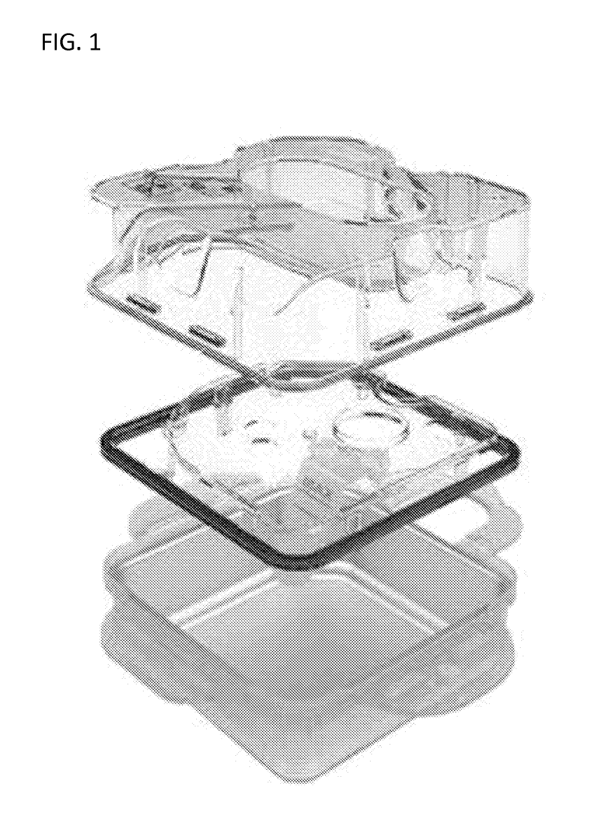

FIG. 1 depicts an assisted breathing apparatus according to one particular, non-limiting embodiment in which the present invention in its various embodiments may be implemented.

The present invention is used in conjunction with a positive airway pressure apparatus and associated methods for use in conjunction with positive airway pressure therapies. The figures generally illustrate embodiments of a positive airway pressure apparatus including aspects of the present invention. The particular exemplary embodiments of the positive airway pressure apparatus illustrated in the figures have been chosen for ease of explanation and understanding of various aspects of the present invention. These illustrated embodiments are not meant to limit the scope of coverage but instead to assist in understanding the context of the language used in this specification and the appended claims. Accordingly, variations of positive airway pressure apparatus for use in positive airway pressure therapies that are different from the illustrated embodiments may be encompassed by the appended claims.

A positive airway pressure apparatus in accordance with aspects of the present invention include at least a housing, a blower, a humidifier, tubing and a mask. The positive airway pressure apparatus is configured to position a mask in communication with the airways of a user to provide pressurized air and/or other gases for positive airway pressure therapy. The therapy may provide continuous or variable positive airway pressure to the airways of the user as will be recognized by those skilled in the art upon review of the present disclosure.

The humidifier unit of a CPAP device is configured to release moisture into the pressurized air passing through the air delivery passage to humidify the pressurized air delivered to the user. The moisture provided by the humidifier may be in the form of water vapor, liquid water droplets, mist, micro-droplets, fog, or various combinations of liquid water and water vapor. The pressurized air may be humidified for therapy, comfort, or other reasons, as will be recognized by those skilled in the art upon review of the present disclosure. The humidifier may be secured to or within the housing of the CPAP device. The humidifier includes at least a humidifier reservoir and, in certain configurations, a humidifier pump, humidifier heater and/or a nebulizer. The humidifier may add moisture and/or therapeutic agents to the air delivered to the user. For exemplary purposes, the humidifier includes a humidifier reservoir secured within the housing into which reservoir or chamber a pre-filled, disposable water container of the present invention is placed. In some embodiments the device, such as a CPAP device, further comprises a UV light source configured to illuminate the hydrating reservoir or chamber, including a disposable pre-filled water container of the present invention. The use of UV illumination is used to sterilize the water in the reservoir or chamber of existing hydration reservoirs or in a pre-filled, disposable water container of the present invention. The UV light source can be configured to illuminate when the CPAP device on (or when it is not in use, for example using a timer). An aperture accessible by a user may be provided on the humidifier housing for placing the pre-filled, disposable water container within the humidifier reservoir. The pre-filled, disposable water container seated in the humidifier reservoir typically contains distilled water and/or therapeutic agents and/or aromatic substance to be introduced as part of the user's therapy. The pre-filled, disposable water containers of the present invention may contain distilled water in volumes of at least 100 ml, at least 150 ml, at least 200 ml, at least 250 ml, at least 300 ml, at least 350 ml, at least 400 ml, at least 450 ml, or at least 500 ml. In certain example embodiments, the disposable water container may be designed and configured to fit generally with a variety of existing CPAP devices. Alternatively, the disposable water container may be specifically designed and configured to fit one or more specific CPAP devices. That is, the disposable water container may be custom fitted to one or more specific CPAP devices.

Depending on the particular configuration, the humidifier reservoir may be in fluid communication with a humidification port to introduce moisture from the pre-filled, disposable water container within the humidifier reservoir into the pressurized air produced by the blower. The humidifier reservoir, with the pre-filled, disposable water container seated within the reservoir, may be resident on the housing or may be positioned remote from the housing as will be recognized by those skilled in art upon review of the present disclosure. The humidification port may be located anywhere along the air delivery passage and/or within aspects of the air pressurizing assembly.

In certain example embodiments, the disposable water container may include a vent or valve, such as a one-way vent or valve, which permits air into the chamber of the water container.

An adaptor or connection means may be used to connect the pre-filled, disposable water container with the humidifier reservoir of the CPAP device. In some embodiments, the nebulizer is connected in series with the airstream. In some embodiments, the nebulizer is connected as a Y to the air stream, such that the nebulized droplets are introduced into the airstream at the Y junction.

A humidifier heater may heat the water and/or therapeutic agents and/or aromatic substance contained within the pre-filled, disposable water container seated in the reservoir to facilitate their introduction into the pressurized air within the air delivery passage for the comfort or therapy of the user. In certain aspects, the humidifier heater may provide a rate of evaporation adequate to humidify the air delivered to the user.

In an alternative embodiment, an ultrasonic or jet nebulizer may be used to aerosolize the hydrating water with or without therapeutic agents and/or aromatic substances. The moisture from the pre-filled, disposable water containers seated within the humidifier reservoir is mixed with the aerosolized therapeutic agents and/or aromatic substances, and delivered to the user via the air delivery passage. The use of a nebulizer may be in addition to or as an alternative to the heated water chamber. If in addition to a heated water chamber, after the humidified air passes out of the water chamber, it then passes through the nebulizer and mixes with the aerosolized therapeutic agents and/or aromatic substances. If the nebulizer is an alternative to the heated water chamber, the air from the CPAP device goes directly to the nebulizer for hydration after filling with the pre-filled distilled water, and can be used with or without therapeutic agents and/or aromatic substances. In some embodiments of the invention, the nebulizer is a piezoelectric device (for example similar to an ultrasonic cleaner or an inkjet head) configured to deliver small microdroplets of distilled water and/or therapeutics capable of infiltrating deep into the lungs. Small microdroplets are particularly useful in delivering substances, such as therapeutics, for example drugs and/or surfactants deep into the alveoli of the lungs.

After the contents of the pre-filled, disposable water container are depleted, the disposable container may be discarded, thus eliminating any need to clean or dry the humidifier after use for subsequent use or packing.

Alternatively, the pre-filled, disposable water container may be disposed of after emptying its contents into the humidifier reservoir.

While the technology has been described in connection with several examples, it is to be understood that the technology is not to be limited to the disclosed examples, but on the contrary, is intended to cover various modifications and equivalent arrangements included within the spirit and scope of the technology. Also, the various examples described above may be implemented in conjunction with other examples, e.g., one or more aspects of one example may be combined with aspects of another example to realize yet other examples. Further, each independent feature or component of any given assembly may constitute an additional example. In addition, while the technology has particular application to patients who suffer from respiratory disorders and/or OSA, it is to be appreciated that patients who suffer from other illnesses (e.g., diabetes, cystic fibrosis, COPD, asthma, pulmonary infections, pneumonia, lung cancer, morbid obesity, stroke, bariatric surgery, Alzhiemer's etc.) can derive benefit from the above teachings. Moreover, the above teachings have applicability with patients and non-patients alike in non-medical applications.

Aromatic Substances

The fragrance according to this disclosure may comprise one or more fragrant materials or materials that provide chemically active vapors. In one embodiment, the fragrance can comprise and/or include volatile, fragrant compounds including, but not limited to natural botanic extracts, essences, fragrance oils, and so forth. As is known in the art, many essential oils and other natural plant derivatives contain large percentages of highly volatile scents. In this regard, numerous essential oils, essences, and scented concentrates are commonly available from companies in the fragrance and food businesses.

Exemplary oils and extracts include, but are not limited to, those derived from the following plants: almond, amyris, anise, armoise, bergamot, cabreuva, calendula, canaga, cedar, chamomile, coconut, eucalyptus, fennel, jasmine, juniper, lavender, lemon, lemongrass, orange, palm, peppermint, quassia, rosemary, thyme, and so forth.

The fragrances may also include the essential oils such as elecampane root oil, amyris oil, angelica seed oil, angelica root oil, aniseed oil, araucaria oil, arnica blossom oil, artemisia oil, atractylis oil, valerian oil, basil oil, bay oil, bergamot oil, birch tar oil, bitter almond oil, savory oil, boldo leaf oil, buchu leaf oil, cabreuva oil, cascarilla oil, champak blossom oil, cistus oil, costus root oil, cubebs oil, davana oil, dill oil, dill seed oil, noble fir oil, noble fir cone oil, elemi oil, tarragon oil, eucalyptus oil, fennel oil, pine needle oil, galbanum oil, galangal root oil, geranium oil, ginger grass oil, grapefruit oil, guaiac oil, gurjun balsam oil, helichrysum oil, ho oil, ginger oil, iris oil, cajeput oil, calamus oil, chamomile oil, camphor oil, kananga oil, cardamom oil, carrot seed oil, cassia oil, spruce needle oil, conifer oil, copaiba balsam oil, coriander oil, spearmint oil, caraway oil, cumin oil, lavender oil, leleshwa oil, lemongrass oil, lovage root oil, lime oil, Litsea cubeba oil, laurel leaf oil, mace oil, marjoram oil, mandarin oil, balm oil, mint oil, musk grain oil, myrrh oil, myrtle oil, clove oil, neroli oil, niaouli oil, olibanum oil, oregano oil, orange oil, osmanthus blossom oil, palma rosa oil, passion fruit oil, patchouli oil, peru balsam oil, parsley seed oil, parsley leaf oil, petitgrain oil, pepper oil, peppermint oil, pimento oil, pine oil, pennyroyal oil, rue oil, rosewood oil, rose oil, rosemary oil, savin oil, sage oil, sandalwood oil, sassafras oil, yarrow oil, Schinus molle oil, celery oil, aspic oil, star anise oil, tagetes oil, tea tree oil, terpentine oil, thuja oil, thyme oil, verbena oil, vetiver oil, juniper berry oil, wine yeast oil, wormwood oil, wintergreen oil, ylang ylang oil, ysop oil, zdravetz oil, cedar wood oil, cinnamon oil, cinnamon leaf oil, citronella oil, lemon oil and cypress oil.

The fragrances also include extracts, resinoids, and balsams, such as tree moss extracts, benzoin resin, boronia, Canada balsam, cassie flower extract, rosin, copaiba balsam, dammar resin, daphne extract, oak moss extracts, elemi resinoid, fig leaf absolute, galbanum, gurjun balsam, orris butter, jasmine, labdanum resinoid, longoza extract, mastic, myrrh, narcissus extracts, olibanum (frankincense), opoponax, peru balsam, storax balsam, tolu balsam, tonka bean extract, tuberose extract, vanilla extract, and violet. Extracts of animal origin may also be included among these: amber grease, castoreum, musk, and civet.

The fragrances also include individual or natural or synthetic odorants (“uniform odorants”) of the type of the esters, ethers, alcohols, aldehydes, ketones, hydrocarbons, terpenes and cyclic compounds. They are known to those skilled in the art from relevant handbooks, e.g.: S. Arctander: “Perfume and Flavour Chemicals”, Montclair, (1969) or K Bauer, D. Garbe: “Common Fragrance and Flavor Materials”, VCH, Weinheim (1985). As fragrances it is also possible, it will be appreciated, to use mixtures of the aforementioned substances (“perfume compositions”).

Therapeutic Agents

Substances (e.g. drugs, therapeutic agents, active agents, etc.) that may be delivered as described herein include but are not limited to various agents, drugs, compounds, and compositions of matter or mixtures thereof that provide some beneficial pharmacologic effect. The particles of the invention broadly encompass substances including “small molecule” drugs, vaccines, vitamins, nutrients, aroma-therapy substances, and other-beneficial agents. As used herein, the terms further include any physiologically or pharmacologically active substance that produces a localized or systemic effect in a patient, i.e. the agent may be active in the lung, or may be delivered to the lung as a gateway to systemic activity.

In some embodiments, the site of action of the substance that is delivered may be the lung itself. Examples of such agents include but are not limited to agents for anesthesia; treatments for asthma or other lung conditions; anti-viral, anti-bacterial or anti-fungal agents; anti-cancer agents; α-1 antitrypsin and other antiproteases (for congenital deficiencies), rhDNAse (for cystic fibrosis), and cyclosporine (for lung transplantation), vaccines, proteins and peptides, etc. Other examples include bronchodilators including albuterol, terbutaline, isoprenaline and levalbuterol, and racemic epinephrine and salts thereof; anti-cholinergics including atropine, ipratropium bromide, tiatropium and salts thereof; expectorants including dornase alpha (Pulmozyme®) (used in the management of cystic fibrosis); cysteine, cystamine, or cysteamine (used in treating cystic fibrosis); mannitol (a mucolytic used in treating cystic fibrosis); corticosteroids such as budesonide, triamcinolone, fluticasone; prophylactic anti-asthmatics such as sodium cromoglycate and nedocromil sodium; anti-infectives such as the antibiotic gentamicin and the anti-protozoan pentamidine (used in the treatment of Pneumocystis carinii pneumonia), and the antiviral agent ribavirin, used to treat respiratory syncytial virus e.g. in young children and infants. In another example, it may be desirable to target areas for the lungs to delivery of therapeutic agents. In this example, anti-infective agents may be required to treat localized lung infections within the airways. Targeting to specific regions within the lung and delivering drug aerosols with high deposition efficiencies is possible with this invention. Once a target region has been identified (through clinical examination), an aerosol would be produced that would have a final particle size suitable for deposition in that region. In some embodiments, a therapeutic agent is a treatment for CF, such as a surfactant or an agent to remove bacterial biofilms, such as Nitric Oxide (for example as a carrier gas or component thereof) or others. In some examples for treating CF, the drug is Pulmozyme®, which is a synthetic protein that breaks down excess DNA in the pulmonary secretions of people with cystic fibrosis.

Anti-Infective Agents

Examples of anti-infective agents, whose class or therapeutic category is herein understood as comprising compounds which are effective against bacterial, fungal, and viral infections, i.e. encompassing the classes of antimicrobials, antibiotics, antifungals, antiseptics, and antivirals, are penicillins, including benzylpenicillins (penicillin-G-sodium, clemizone penicillin, benzathine penicillin G), phenoxypenicillins (penicillin V, propicillin), aminobenzylpenicillins (ampicillin, amoxycillin, bacampicillin), acylaminopenicillins (azlocillin, mezlocillin, piperacillin, apalcillin), carboxypenicillins (carbenicillin, ticarcillin, temocillin), isoxazolyl penicillins (oxacillin, cloxacillin, dicloxacillin, flucloxacillin), and amiidine penicillins (mecillinam); cephalosporins, including cefazolins (cefazolin, cefazedone); cefuroximes (cerufoxim, cefamdole, cefotiam), cefoxitins (cefoxitin, cefotetan, latamoxef, flomoxef), cefotaximes (cefotaxime, ceftriaxone, ceftizoxime, cefmenoxime), ceftazidimes (ceftazidime, cefpirome, cefepime), cefalexins (cefalexin, cefaclor, cefadroxil, cefradine, loracarbef, cefprozil), and cefiximes (cefixime, cefpodoxim proxetile, cefuroxime axetil, cefetamet pivoxil, cefotiam hexetil), loracarbef, cefepim, clavulanic acid/amoxicillin, Ceftobiprole; synergists, including beta-lactamase inhibitors, such as clavulanic acid, sulbactam, and tazobactam; carbapenems, including imipenem, cilastin, meropenem, doripenem, tebipenem, ertapenem, ritipenam, and biapenem; monobactams, including aztreonam; aminoglycosides, such as apramycin, gentamicin, amikacin, isepamicin, arbekacin, tobramycin, netilmicin, spectinomycin, streptomycin, capreomycin, neomycin, paromoycin, and kanamycin; macrolides, including erythromycin, clarythromycin, roxithromycin, azithromycin, dithromycin, josamycin, spiramycin and telithromycin; gyrase inhibitors or fluroquinolones, including ciprofloxacin, gatifloxacin, norfloxacin, ofloxacin, levofloxacin, perfloxacin, lomefloxacin, fleroxacin, garenoxacin, clinafloxacin, sitafloxacin, prulifloxacin, olamufloxacin, caderofloxacin, gemifloxacin, balofloxacin, trovafloxacin, and moxifloxacin; tetracyclins, including tetracyclin, oxytetracyclin, rolitetracyclin, minocyclin, doxycycline, tigecycline and aminocycline; glycopeptides, inlcuding vancomycin, teicoplanin, ristocetin, avoparcin, oritavancin, ramoplanin, and peptide 4; polypeptides, including plectasin, dalbavancin, daptomycin, oritavancin, ramoplanin, dalbavancin, telavancin, bacitracin, tyrothricin, neomycin, kanamycin, mupirocin, paromomycin, polymyxin B and colistin; sulfonamides, including sulfadiazine, sulfamethoxazole, sulfalene, co-trimoxazole, co-trimetrol, co-trimoxazine, and co-tetraxazine; azoles, including clotrimazole, oxiconazole, miconazole, ketoconazole, itraconazole, fluconazole, metronidazole, tinidazole, bifonazol, ravuconazol, posaconazol, voriconazole, and ornidazole and other antifungals including flucytosin, griseofluvin, tonoftal, naftifin, terbinafin, amorolfin, ciclopiroxolamin, echinocandins, such as micafungin, caspofungin, anidulafungin; nitrofurans, including nitrofurantoin and nitrofuranzone; polyenes, including amphotericin B, natamycin, nystatin, flucocytosine; other antibiotics, including tithromycin, lincomycin, clindamycin, oxazolindiones (linzezolids), ranbezolid, streptogramine A+B, pristinamycin aA+B, Virginiamycin A+B, dalfopristin/qiunupristin (Synercid), chloramphenicol, ethambutol, pyrazinamid, terizidon, dapson, prothionamid, fosfomycin, fucidinic acid, rifampicin, isoniazid, cycloserine, terizidone, ansamycin, lysostaphin, iclaprim, mirocin B17, clerocidin, filgrastim, and pentamidine; antivirals, including aciclovir, ganciclovir, birivudin, valaciclovir, zidovudine, didanosin, thiacytidin, stavudin, lamivudin, zalcitabin, ribavirin, nevirapirin, delaviridin, trifluridin, ritonavir, saquinavir, indinavir, foscarnet, amantadin, podophyllotoxin, vidarabine, tromantadine, and proteinase inhibitors; plant extracts or ingredients, such as plant extracts from chamomile, hamamelis, echinacea, calendula, papain, pelargonium, essential oils, myrtol, pinen, limonen, cineole, thymol, mentol, alpha-hederin, bisabolol, lycopodin, vitapherole; wound healing compounds including dexpantenol, allantoin, vitamins, hyaluronic acid, alpha-antitrypsin, anorganic and organic zinc salts/compounds, interferones (alpha, beta, gamma), tumor necrosis factors, cytokines, interleukins.

Active Agents

Additional active agents may be selected from, for example, anti-Alzheimer's disease agents, hypnotics and sedatives, tranquilizers, anticonvulsants, muscle relaxants, antiparkinson agents (dopamine agonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents (particularly those that target lung cancer), antithrombotic agents, antihypertensives, cardiovascular drugs, antiarrhythmics, antioxicants, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically. The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like. In specific examples, an “agent” is an agent for the treatment of Alzheimer's disease and/or OSA (obstructive sleep apnea). In one specific example, the anti-Alzheimer's agent is Donepezil, which has been shown to improve obstructive sleep apnea and Alzheimer disease, see Moraes et al., CHEST. 2008;133(3):677-683.

Examples of other active agents suitable for use in this invention include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-I receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosphonates, respiratory syncytial virus antibody, cystic fibrosis transmembrane regulator (CI-TR) gene, deoxyreibonuclease (Dnase), bactericidal/permeability increasing protein (BPI), anti-CMV antibody, and 13-cis retinoic acid, and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof. Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense and inhibitory RNA. Further, an active agent may comprise live attenuated or killed viruses suitable for use as vaccines. Other useful drugs include those listed within the Physician's Desk Reference (most recent edition).

An active agent for delivery or formulation as described herein may be an inorganic or an organic compound, including, without limitation, drugs which act on: the lung, the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Frequently, the active agent acts in or on the lung.

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of hygroscopic excipient contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

In addition to one or more active agents, the aerosol particles/droplets may optionally include one or more pharmaceutical excipients that are suitable for pulmonary administration. These excipients, if present, are generally present in the composition in amounts ranging from about 0.01% to about 95% percent by weight, preferably from about 0.5 to about 80%, and more preferably from about 1 to about 60% by weight. Preferably, such excipients serve to further improve the features of the active agent composition, for example by improving the handling characteristics of powders, such as flowability and consistency, and/or facilitating manufacturing and filling of unit dosage forms. One or more excipients may also be provided to serve as bulking agents when it is desired to reduce the concentration of active agent in the formulation. Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers. The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-p-cyclodextrin and sulfobutylether-β-cyclodextrin), polyethylene glycols, and pectin. The particles may further include inorganic salts, antimicrobial agents (for example benzalkonium chloride), antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations).

General

To facilitate an understanding of the principles and features of the various embodiments of the invention, various illustrative embodiments are explained above. Although exemplary embodiments of the invention are explained in detail, other embodiments are contemplated. Accordingly, it is not intended that the invention is limited in its scope to the details of construction and arrangement of components set forth in the description or illustrated in the drawings.

Also, in describing the exemplary embodiments, terminology will be resorted to for the sake of clarity. Each term contemplates its broadest meaning as understood by those skilled in the art and includes all technical equivalents that operate in a similar manner to accomplish a similar purpose.

The mention of one or more method steps does not preclude the presence of additional method steps or intervening method steps between those steps expressly identified.

The materials described as making up the various elements of the invention are illustrative and not restrictive. Many suitable materials that would perform the same or a similar function as the materials described herein are encompassed within the scope of the invention. Such other materials not described herein can include, but are not limited to, for example, materials that are developed after the time of the development of the invention.

The example components, construction, and methods described in the embodiments presented previously are illustrative, and, in alternative embodiments, certain components can be placed in a different position, combined with another component, and/or omitted entirely, and/or certain additional components can be added, without departing from the scope and spirit of various embodiments as defined in the claims, the scope of which is to be accorded the broadest interpretation so as to encompass such alternatives.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

1. A method of treating a subject with a disease or condition, comprising:
providing a disposable water container, wherein the disposable water container comprises an agent for the treatment of the disease or condition;
placing the disposable water container in close proximity to an air intake of an assisted breathing unit;
passing air or gas through a flow path having an airstream of the assisted breathing unit wherein vapor and/or droplets from the disposable water container comprising the agent are passed into the air stream, thereby treating the subject.

2. The method of claim 1, wherein the the agent comprises an agent for the treatment of respiratory disorders, and agent for the treatment of Alzhiemer's disease, an ageint for the treatment of chronic obstructive pulmonary disease, anesthesia agents, cystic fibrosis treatments, nucleic acid molecules, anti-pain agents, anti-inflammation agents, anti-depressants, mood altering drugs, anti-viral agents, anti-bacterial agents, anti-fungal agents, anti-cancer agents, hormones, benzodiazepines, calcitonin, mucolytics, chemotherapies, and antiinfectives.

3. The method of claim 1, wherein the disease or condition comprises Alzhiemer's disease.

4. The method of claim 3, wherein the agent comprises Donepezil.

5. The method of claim 4, wherein the agent comprises a biofilm, a surfactant, or pulmozyme,

6. The method of claim 1, wherein the disease or condition comprises cystic fibrosis.

7. The method of claim 1, wherein the disease or condition comprises chronic obstructive pulmonary disease.

8. The method of claim 1, wherein the disease or condition comprises asthma.

9. The method of claim 1, wherein in the agent is nebulized.

10. The method of claim 1, wherein the assisted breathing unit comprises a piezoelectric droplet generator.

11. The method of claim 1, where in the assisted breathing unit comprises an ultra violet light source to sterilize the water.

12. The method of claim 1, wherein the assisted breathing unit comprises a nebulizer and the nebulizer is connected parallel or in series with the airstream to introduce droplets into the airstream.

13. A method of providing a therapeutic agent to a subject, the method comprising: placing a disposable water container in close proximity to the air intake of an assisted breathing unit; providing a therapeutic agent to the water container; passing air or gas through the flow path; evaporating a portion of the therapeutic agent into air being uptaken by the air intake; and providing the uptaken air to a subject.

14. The method of claim 13, wherein the therapeutic agent is selected from the group consisting of agents for the treatment of respiratory disorders, anesthesia agents, nucleic acid molecules, anti-pain agents, anti-inflammation agents, anti-depressants, mood altering drugs, anti-viral agents, anti-bacterial agents, anti-fungal agents, anti-cancer agents, hormones, benzodiazepines, calcitonin, mucolytics, chemotherapies, and antiinfectives.

15. A disposable container for placement within the hydrating chamber of an assisted breathing unit.

16. The disposable container of claim 15, wherein the disposable container is pre-filled with water.

17. The disposable container of claim 16, wherein the water is distilled water.

18. The disposable container of claim 17, wherein the pre-filled, distilled water is in a volume of approximately 100 to 400 ml.

19. The disposable container of claim 15, further comprising an aromatic substance.

20. The disposable container of claim 19, wherein the aromatic substance is selected from the group consisting of natural botanic extracts, essences, fragrance oils, and essential oils.

21. The disposable container of claim 15 claim, further comprising a therapeutic agent.

22. The disposable container of claim 21, wherein the therapeutic agent one or more of agents for the treatment of respiratory disorders, anesthesia agents, nucleic acid molecules, anti-pain agents, anti-inflammation agents, anti-depressants, mood altering drugs, anti-viral agents, anti-bacterial agents, anti-fungal agents, anti-cancer agents, hormones, benzodiazepines, calcitonin, mucolytics, chemotherapies, and antiinfectives.

23. (canceled)

24. A disposable water container for placement within a compartment of an assisted breathing unit, the water container comprising a closed top surface with a top fill aperture and walls sized to fit within the water chamber of an assisted breathing unit.

25. The disposable water container of claim 24, further comprising a heat-resistant bottom surface.

26. (canceled)

27. A kit containing more than two disposable water containers of claim-15.

28. The kit of claim 27, wherein the disposable water containers are packaged together in a group of six units.

29. The kit of claim 27, further comprising a non-volatile antiseptic.

30. The kit of claim 27, further comprising an aromatic substance.

31. The kit of claim 27, further comprising a therapeutic agent.

32. A method of providing an aromatic substance to a subject, the method comprising: placing a disposable water container in close proximity to the air intake of an assisted breathing unit; providing an aromatic substance to the water container; passing air or gas through the flow path; evaporating a portion of the aromatic or aroma therapy substance into air being uptaken by the air intake; and providing the uptaken air to a subject.

33. The method of claim 32, wherein the aromatic substance comprises one or more of natural botanic extracts, essences, fragrance oils, and essential oils.
